SEQUENCE LISTING (2 sequences)

SEQ ID NO: 1 - DNA, 20 bp, synthetic construct
  source 1..20: mol_type = other DNA
gtggtgtctcggagcgatag

SEQ ID NO: 2 - DNA, 20 bp, synthetic construct
  source 1..20: mol_type = other DNA
taacgaccgcgtgagtcaaa

(19) United States
(12) Patent Application Publication
(10) Pub. No.: US 2026/0201326 A1
(43) Pub. Date: Jul. 16, 2026

(54) USE OF CHICK EMBRYO EXTRACT IN PROMOTING SYNTHESIS OF LACTOFERRIN BY MAMMARY EPITHELIAL CELLS

(71) Applicant: BEIJING SANYUAN FOODS CO., LTD., Beijing (CN)

(72) Inventors: Lijun CHEN, Beijing (CN); Changjun LUO, Beijing (CN); Zhenzhen ZHANG, Beijing (CN); Mengna JING, Beijing (CN); Jiahui REN, Beijing (CN); Tiemin JIANG, Beijing (CN); Junying ZHAO, Beijing (CN); Weicang QIAO, Beijing (CN)

(73) Assignee: BEIJING SANYUAN FOODS CO., LTD., Beijing (CN)

(21) Appl. No.: 19/321,252

(22) Filed: Sep. 7, 2025

(30) Foreign Application Priority Data
Jan. 10, 2025 (CN) 2025100392580

(51) Int. Cl. C12N 5/071

(52) U.S. Cl. CPC C12N 5/0631; C12N 2500/84

(57) ABSTRACT

Provided is use of a chick embryo extract in promoting synthesis of lactoferrin by mammary epithelial cells. Experiments prove that adding the chick embryo extract to a medium for culturing the mammary epithelial cells can increase expression of lactoferrin-encoding genes in the mammary epithelial cells, and promote secretion of lactoferrin, thus improving efficiency of biosynthesis of lactoferrin, and solving technical problems in the prior art that lactoferrin synthesis is costly and inefficient, and is difficult to meet market demand for the lactoferrin.

### CROSS-REFERENCE TO RELATED APPLICATIONS

The present disclosure claims priority to Chinese Patent Application No. 202510039258.0, entitled “USE OF CHICK EMBRYO EXTRACT IN PROMOTING SYNTHESIS OF LACTOFERRIN BY MAMMARY EPITHELIAL CELLS”, filed on Jan. 10, 2025, the entire contents of which are incorporated herein by reference.

### REFERENCE TO AN ELECTRONIC SEQUENCE LISTING

The contents of the electronic sequence listing (FOUND-P-004-US.xml; Size: 2,799 bytes; and Date of Creation: Sep. 4, 2025) is herein incorporated by reference in its entirety.

### TECHNICAL FIELD

The present disclosure relates to technical field of in vitro culture, and particularly to use of a chick embryo extract in promoting synthesis of lactoferrin by mammary epithelial cells.

### BACKGROUND ART

Chick embryo egg, also called as “balot (balut)”, refers to unbroken edible fertilized chick egg, containing a variety of proteins, amino acids, growth factors, minerals, vitamins, etc. Chick embryo egg is not only a natural dietary supplement, known as “a popular tonic”, but also can provide some critical growth factors for cell culture, and is widely used for supplementing growth culture media of various cell types, such as neural crest stem cells, chick embryo fibroblasts, skeletal muscle stem cells, and glomerular cells. It has potential health promoting, medicinal and biological values.

LF (lactoferrin) is a multifunctional glycoprotein widely existing in milk, saliva and other biological fluids, and has important biological activities such as antibiosis, antivirus, immunoregulation and iron absorption promotion.

At present, research directions at home and abroad mostly focus on the function aspect of LF, and a scientific problem of how to improve LF yield is rarely reported. Given a substantial increase in domestic demand for LF, how to break free from restriction of LF raw materials on China has become an urgent problem.

Although several methods for extracting and purifying LF have been developed in the prior art, many challenges remain in terms of its expression level and activity. Existing production methods typically rely on complex fermentation technologies, with high costs and low efficiency, and are difficult to meet the market demand for LF. In addition, natural LF is limited in source and is difficult to produce on a large scale.

Therefore, an urgent need exists for a novel method and formulation to improve an expression level of LF, reduce production costs, and preserve bioactivities thereof.

In view of this, the present disclosure is specifically proposed.

### SUMMARY

A first objective of the present disclosure is to provide use of a chick embryo extract in promoting synthesis of lactoferrin by mammary epithelial cells, so as to solve the technical problems in the prior art that lactoferrin synthesis is inefficient, and it is difficult to meet market demand for LF.

A second objective of the present disclosure is to provide a composition for promoting synthesis of lactoferrin by mammary epithelial cells.

A third objective of the present disclosure is to provide a method for promoting lactoferrin synthesis by mammary epithelial cells in vitro.

In order to achieve the above objectives of the present disclosure, following technical solutions are particularly adopted.

In the first aspect, the present disclosure provides use of a chick embryo extract in promoting synthesis of lactoferrin by mammary epithelial cells.

Further, the promoting synthesis of lactoferrin by mammary epithelial cells includes improving an expression level of lactoferrin-encoding genes and/or increasing a lactoferrin content.

Further, the mammary epithelial cells are derived from mammals.

Further, the mammals include any one selected from the group consisting of humans, cattle, sheep, camels, horses, donkeys, reindeer, alpacas, and llamas.

In the second aspect, the present disclosure provides a composition for promoting synthesis of lactoferrin by mammary epithelial cells, including 0.25-0.5 mL of a chick embryo extract per 100 mL of a medium.

Further, the composition includes 0.25 mL of the chick embryo extract per 100 mL of the medium.

Further, the medium is a mammary epithelial cell medium.

Further, the mammary epithelial cell medium includes a DMEM medium and a fetal bovine serum.

In the third aspect, the present disclosure provides a method for promoting synthesis of lactoferrin by mammary epithelial cells in vitro, using the above composition for culturing the mammary epithelial cells.

In some embodiments, when the chick embryo extract is added in an amount of 0.25 mL per 100 mL of the medium, a culturing duration is at least 4 days; and

when the chick embryo extract is added in an amount of 0.5 mL per 100 mL of the medium, the culturing duration is at least 6 days.

The present disclosure provides the use of the chick embryo extract in promoting synthesis of lactoferrin by the mammary epithelial cells. Experiments prove that adding the chick embryo extract (CEE) to the medium for culturing the mammary epithelial cells can increase expression of the lactoferrin-encoding genes in the mammary epithelial cells, and promote the secretion of lactoferrin (LF), thus improving the efficiency of biosynthesis of lactoferrin, and solving the technical problems in the prior art that the lactoferrin synthesis is costly and inefficient, and is difficult to meet the market demand for the lactoferrin.

### BRIEF DESCRIPTION OF THE DRAWINGS

In order to more clearly illustrate technical solutions in embodiments of the present disclosure or the prior art, drawings that need to be used in the description of the embodiments or the prior art will be briefly introduced below. Obviously, the drawings in the following description show some embodiments of the present disclosure, and those ordinarily skilled in the art still could obtain other drawings according to these drawings without using any inventive efforts.

FIG. 1 shows D1-D6 growth charts of MAC-T cells treated with different amounts of CEE added provided by Example 2 of the present disclosure;

FIG. 2 shows CK18 immunofluorescent staining images of MAC-T cells treated without CEE and with 0.25% CEE provided by Example 3 of the present disclosure;

FIG. 3 shows microscopic images of MAC-T cells treated without CEE and with 0.25% CEE provided by Example 4 of the present disclosure;

FIG. 4 is a count statistical chart of MAC-T cells treated without CEE and with 0.25% CEE provided by Example 4 of the present disclosure;

FIG. 5 is a statistical chart of LF contents of MAC-T cells treated without CEE and with 0.25% of CEE provided by Example 5 of the present disclosure;

FIG. 6 is a statistical chart of LF gene expression level in MAC-T cells treated without CEE and with 0.25% of CEE provided by Example 6 of the present disclosure;

FIG. 7 shows microscopic images of MCF-10A cells treated without CEE and with 0.25% CEE provided by Example 8 of the present disclosure;

FIG. 8 is a count statistical chart of MCF-10A cells treated without CEE and with 0.25% CEE provided by Example 8 of the present disclosure; and

FIG. 9 is a statistical chart of LF contents of MCF-10A cells treated without CEE and with 0.25% CEE provided by Example 9 of the present disclosure.

### DETAILED DESCRIPTION OF EMBODIMENTS

Unless otherwise defined herein, scientific and technical terms used in connection with the present disclosure shall have the meanings that are commonly understood by those ordinarily skilled in the art. The meanings and scopes of the terms should be clear, however, in the event of any latent ambiguity, definitions provided herein take precedent over any dictionary or extrinsic definition. In the present disclosure, use of “or” means “and/or” unless otherwise stated. In addition, use of the term “include (comprise)” and other forms is non-limiting.

Unless otherwise stated, the methods and technologies in the present disclosure are generally carried out according to conventional methods well-known in the art and as described in various general and more specific reference documents, and the reference documents are cited and discussed throughout the present description.

In one aspect, the present disclosure provides use of a chick embryo extract in promoting synthesis of lactoferrin by mammary epithelial cells.

Experiments prove that adding the chick embryo extract (CEE) to a medium for culturing the mammary epithelial cells can increase expression of lactoferrin-encoding genes in the mammary epithelial cells, and promote secretion of lactoferrin (LF), thus improving the efficiency of biosynthesis of lactoferrin, and solving the technical problems in the prior art that the lactoferrin synthesis is costly and inefficient, and is difficult to meet the market demand for the lactoferrin.

In some embodiments, the promoting the synthesis of lactoferrin by mammary epithelial cells includes improving an expression level of lactoferrin-encoding genes and/or increasing a lactoferrin content.

In some embodiments, the mammary epithelial cells are derived from mammals.

Herein, the mammals include animals that are capable of secreting milk for nursing offspring.

Experiments prove that the chick embryo extract can promote the expression of lactoferrin genes in human mammary epithelial cells and bovine mammary epithelial cells, and a synthesis pathway in the mammary epithelial cells involves expression of the lactoferrin genes in the mammary epithelial cells, the chick embryo extract is thus able to facilitate the mammary epithelial cells from mammals in synthesizing lactoferrin. In some embodiments, the mammals include any one selected from the group consisting of humans, cattle, sheep, camels, horses, donkeys, reindeer, alpacas, and llamas.

According to another aspect, the present disclosure further provides a composition for promoting synthesis of lactoferrin by mammary epithelial cells, including 0.25-0.5 mL of a chick embryo extract per 100 mL of a medium.

By adding the chick embryo extract to the medium of the composition, the chick embryo extract can enhance expression of lactoferrin-encoding genes in the mammary epithelial cells, and synthesis of lactoferrin.

Herein, a content of the chick embryo extract may be, but not limited to, 0.25 mL per 100 mL of the medium, 0.27 mL per 100 mL of the medium, 0.3 mL per 100 mL of the medium, 0.33 mL per 100 mL of the medium, 0.35 mL per 100 mL of the medium, 0.38 mL per 100 mL of the medium, 0.4 mL per 100 mL of the medium, 0.43 mL per 100 mL of the medium, 0.45 mL per 100 mL of the medium, 0.48 mL per 100 mL of the medium or 0.5 mL per 100 mL of the medium, and also may be any value in a range of 0.25-0.5 mL per 100 mL of the medium.

In some embodiments, the composition includes 0.25 mL of the chick embryo extract per 100 mL of the medium.

In some embodiments, the medium is a mammary epithelial cell medium.

In some embodiments, the mammary epithelial cell medium includes a DMEM medium and a fetal bovine serum.

Herein, the DMEM medium and the fetal bovine serum can be specifically selected, and penicillin and/or streptomycin can also be added.

In another aspect, the present disclosure further provides a method for promoting synthesis of lactoferrin by mammary epithelial cells in vitro, using the above composition for culturing the mammary epithelial cells.

By adding CEE to the medium for culturing the mammary epithelial cells, the expression of the lactoferrin-encoding genes in the mammary epithelial cells and synthesis of lactoferrin are promoted during the culture.

Experiments revealed that when the chick embryo extract added is added in an amount of 0.25 mL per 100 mL of the medium, from the fourth day of culturing, a significant stable difference occurs to cell amount compared with that without the chick embryo extract. From the sixth day of culturing, a significant difference occurs to the cell amount between cases with 0.5 mL of the chick embryo extract per 100 mL of the medium and without the chick embryo extract. In some embodiments, when the chick embryo extract is added in an amount of 0.25 mL per 100 mL of the medium, a culturing duration is at least 4 days. When the chick embryo extract is added in an amount of 0.5 mL per 100 mL of the medium, the culturing duration is at least 6 days.

Technical solutions in the present disclosure will be clearly and completely described below in combination with the embodiments. Apparently, only some but not all of the embodiments are described. Based on the embodiments in the present disclosure, all of other embodiments obtained by those ordinarily skilled in the art without using any inventive efforts shall fall within the scope of protection of the present disclosure.

Fetal bovine serum (FBS) and DMEM medium were both purchased from Gibco (Grand Island Biological Company).

### Preparation Method for Chick Embryo Extract:

(1) taking 10-day-old specified pathogen-free hatched chick embryo eggs (SPF), where a hatching temperature in an incubator was 37.8° C., and humidity was 60%; cleaning, and soaking in 75% alcohol for 20 min for sterilizing;
(2) under aseptic conditions, placing the chick embryo eggs using sterilized scissors and forceps, with air cell upwards, cracking egg shells at the air cell using the forceps, transferring chick embryo into a beaker, and removing air sac, amniotic sac, and chorioallantoic membrane;
(3) rinsing with a 4° C. sterile pbs solution 4 times until solution was clear, adding an equal amount of a 4° C. sterile hanks solution, and homogenizing with a high-speed homogenizer at 18,000 r/min for 2 min;
(4) separately charging homogenized chick embryo into several small parts and sealing well, followed by leaching in constant temperature culture at 37° C. for 30 min;
(5) after leaching, performing disruption by a non-contact ultrasonic cell disruptor at a temperature 4° C. and power of 1,000 W for 1 h, under treatment conditions of: processing for 30 s, pausing for 30 s, in cyclic repetition;
(6) placing disrupted chick embryo into liquid nitrogen, dissolving at room temperature, and repeatedly freezing and thawing for 2 times; and
(7) centrifuging by a refrigerated centrifuge at 4° C. and 6,000 g for 10 min, collecting supernatant, performing sterile filtration sequentially at 10 μm/3 μm/1 μm/0.45 μm/0.22 μm, and storing in a refrigerator at −80° C. for later use.

### Example 1 Composition for Promoting Synthesis of Lactoferrin by Mammary Epithelial Cells

Formulation of the composition is as listed in Table 1.

TABLE 1

| Group | Component | | | | | |
|---|---|---|---|---|---|---|
| Control | DMEM | 10% FBS | 1% Penicillin | 1% | Without | CEE |
| Group 1 | Medium | | (10000 U/mL) | Streptomycin | | |
| Experimental | | | | (10000 U/mL) | 0.125% | CEE |
| Group 1 | | | | | | |
| Experimental | | | | | 0.25% | CEE |
| Group 2 | | | | | | |
| Experimental | | | | | 0.5% | CEE |
| Group 3 | | | | | | |
| Experimental | | | | | 1% | CEE |
| Group 4 | | | | | | |
| Experimental | | | | | 2% | CEE |
| Group 5 | | | | | | |

Herein, 0.125% CEE means adding 0.125 ml of CEE per 100 ml of the medium, 0.25% CEE means adding 0.25 ml of CEE per 100 ml of the medium, 0.5% CEE means adding 0.5 ml of CEE per 100 ml of the medium, 1% CEE means adding 1 ml of CEE per 100 ml of the medium, 2% CEE means adding 2 ml of CEE per 100 ml of the medium.

### Example 2 Measurement of MAC-T Cell Growth Curves by CCK8 Method

CCK8 method was adopted. 100 μl of 2,000 MAC-T cells were added to each well of a 96-well plate. After 12 h of adherent culture of the cells in a complete medium without CEE, the control group was changed with a complete medium without the CEE as listed in Table 1 (Control Group 1); the experimental groups were changed with a complete medium with 0.125-2% CEE (Experimental Groups 1-5). Every other day, 10 μl of a CCK-8 solution was added to each well. Further incubation was performed for 2 hours in the cell incubator, followed by measurement of absorbance at 450 nm, for 6 consecutive days.

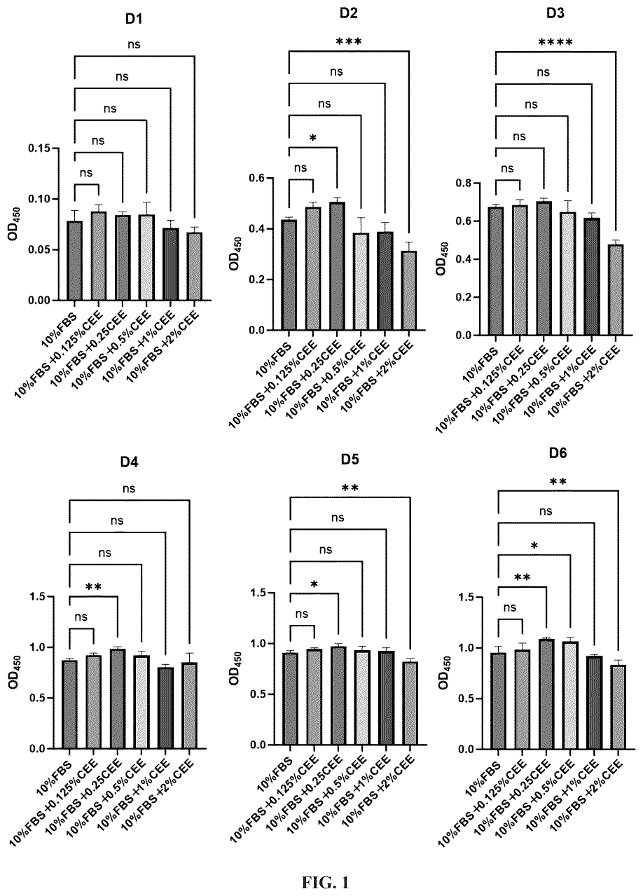

Outcomes are as shown in FIG. 1. Cell growth curves of the experimental groups and the control group followed typical cell growth rules. After adding different proportions of the CEE in the complete medium in the present disclosure, a cell proliferation rate obtained by adding 0.25% CEE was significantly higher than that of the control group on days 2, 4, 5 and 6, and the cell proliferation rate obtained by adding 0.5% CEE was significantly higher than that of the control group on day 6.

### Example 3 CK18 Immunofluorescent Staining Comparison of MAC-T Cells

After 36 h of adherent culture, the MAC-T cells in the control group and the Experimental group 2 were subjected to immunofluorescent staining.

Preparation of PBSTx: to PBS, adding Triton X-100 to render a final concentration of 0.1%, and mixing well, thus obtaining TBSTx.

Preparation of blocking buffer BSA-PBSTx: to 100 ml of PBSTx, adding 5 g of BSA, dissolving and mixing well, thus obtaining BSA-PBSTx.

Preparation of primary antibody dilution buffer: diluting CK18 as antibody 300-fold with the blocking buffer BSA-PBSTx.

Preparation of secondary antibody dilution buffer: diluting secondary antibody 200-fold with blocking buffer BSA-PBSTx.
1. A common clean cover glass was soaked in 70% ethanol for 5 min, washed with a cell culture grade PBS solution three times, and then washed once with a cell culture solution. The cover glass was placed in a six-well plate, and 2.5×10⁵ cells were seeded in each well. After 12 h, the control group was changed with the complete medium without chick embryo, and the experimental group was changed with the complete medium with 0.25% chick embryo. Further culturing was performed for 24 h, to render a cell density of about 50%-80%.
2. Culture solution was discarded, and resultant was cleaned three times with a sterile pbs solution, 5 min each time. 1 ml of 4% paraformaldehyde fixation solution was added to each well, followed by fixation at 37° C. for 10 min.
3. The fixation solution was discarded. Resultant was washed with the pbs solution 3 times, with gentle shaking by hand, 5 min each time, and liquid was absorbed completely.
4. Blocking was done with the blocking buffer BSA-PBSTx at 37° C. for 60 min.
5. The blocking buffer was removed, and 1 ml of diluted primary antibody was added to each well over night at 4° C.
6. After overnight, the primary antibody was removed, and resultant was washed with pbs solution 3 times, 5 min each time, with gentle shaking on each wash.
7. Washing solution was removed, and 1 ml of diluted fluorescently labeled secondary antibody was added and incubated at 37° C. for 1 h in the dark.
8. One drop of an anti-fluorescence quenching mounting medium was placed on a glass slide, and the cover glass with cells attached thereto was placed thereon, trying to avoid air bubbles. The cells were brought into contact with the mounting medium.
9. Fluorescence was observed under a fluorescence microscope.

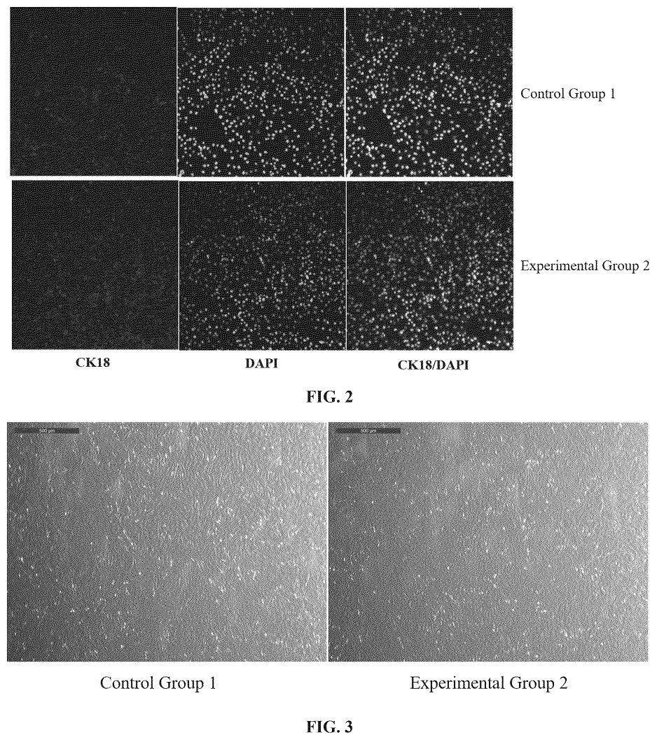

Outcomes are as shown in FIG. 2, and it can be seen from CK18 immunofluorescent staining images that the addition of CEE did not produce apparent changes in morphology and structure of cells.

### Example 4 Microscopic Observation and Cell Counting of MAC-T Cells

MAC-T cells were seeded in six-well plates at 2.5×10⁵ cells per well. After 12 h of culture in complete medium without CEE, the control group was changed with the complete medium without CEE (Control Group 1), and the experimental group was changed with the complete medium with 0.25% CEE (Experimental Group 2). Microscopy and cell counting were performed on the following day.

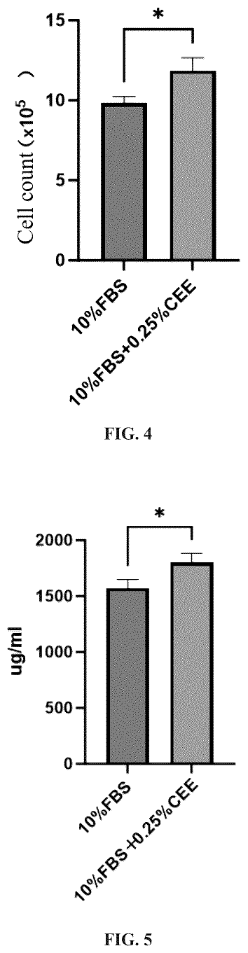

Outcomes are as shown in FIG. 3. After 48 h of culture of the MAC-T cells over-expressing LF using a pancreatic digestion method, cell growth status was good. The experimental group and the control group were full, had a three-dimensional feeling, and exhibited a spindle shape or a triangular shape. As seen from outcomes of cell counting in FIG. 4, the number of cells cultured with 0.25% CEE was larger than that of the control group without CEE.

### Example 5 Detection of LF Expression in MAC-T Cells by ELISA Kit.

MAC-T cells were seeded in six-well plates at 2.5×10⁵ cells per well. After 12 h of culture in complete medium without CEE, the control group was changed with the complete medium without CEE (Control Group 1), and the experimental group was changed with the complete medium with 0.25% CEE (Experimental Group 2). After 12 h of further culture, 20 μl of doxycycline (DOX) induction solution at a concentration of 100 μg/ml was added to each well in the six-well plates. On the third day of culture, the cell medium was pipetted and centrifuged at 1,000×g for 20 min, and supernatant was taken to detect expression condition of bovine LF.
1. Sample loading: Standard wells, to-be-detected sample wells, and blank wells were respectively set. 7 standard wells were set, and added with 100 μL of standards at different concentrations in turn. 100 μL of standard diluent was added to the blank wells. Remaining wells were added with 100 μL of the to-be-detected sample. Microplate was covered by a membrane, followed by incubation at 37° C. for 1 hour.
2. Liquid was discarded, followed by spin drying, without washing.
3. To each well, 100 μL of detection solution A working solution (prepared immediately before use) was added, and the microplate was covered by a membrane, followed by incubation at 37° C. for 1 hour.
4. Liquid in the wells was discarded, and each well was washed with 350 μL of washing solution, and soaked for 1-2 min. The microplate was gently tapped on absorbent paper to remove all liquid from the wells. Plate washing was repeated 3 times. After the final wash, remaining wash buffer was aspirated or decant. The microplate was inverted on the absorbent paper to blot thoroughly all residual liquid in the wells.
5. To each well, 100 μL of detection solution B working solution (prepared immediately before use) was added, and the microplate was covered by a membrane, followed by incubation at 37° C. for 30 min.
6. Liquid in the wells was discarded, followed by spin drying, and 5 times of plate washing, by the same method as in step 4.
7. To each well, 90 μL of TMB backing solution was added, and the microplate was covered by a membrane, followed by color development at 37° C. in the dark for 10 min. When there was an apparent gradient of blue color in the first 3-4 wells of the standard wells, and a gradient of the last 3-4 wells was not apparent, reaction was terminated.
8. To each well, 50 μL of stop solution was added to terminate the reaction, and at this time, blue color was turned to yellow color immediately. An order of adding the stop solution should be as similar as possible to an order of adding the backing solution.
9. After ensuring no water drop at the bottom of the microplate and no air bubble in the wells, the absorbance of each well was immediately measured at a wavelength of 450 nm by a microplate reader, and then the absorbance was converted into LF concentration.

Outcomes are as shown in FIG. 5, and it can be seen that the LF content of the cells in the experimental group with 0.25% CEE was high than that in the control group.

### Example 6 RT-qPCR Detection of LF Gene Expression in MAC-T Cells

MAC-T cells were seeded in six-well plates at 2.5×10⁵ cells per well. After 12 h of culture in complete medium without CEE, the control group was changed with the complete medium without CEE (Control Group 1), and the experimental group was changed with the complete medium with 0.25% CEE (Experimental Group 2). After 12 h of further culture, 20 μl of DOX induction solution at a concentration of 100 μg/ml was added to each well in the six-well plates. RNA was extracted the following day, and subjected to reverse transcription to cDNA, and then qPCR amplification was performed. A target fragment length was 774 bp. Amplification primer information is as listed in Table 2. A reaction system is as listed in Table 3. Reaction conditions are as listed in Table 4.

TABLE 2

| Primer | Sequence 5′~3′ |
|---|---|
| N3-001040-3-F | GTGGTGTCTCGGAGCGATAG |
| | (SEQ ID NO. 1) |
| N3-001040-3-R | TAACGACCGCGTGAGTCAAA |
| | (SEQ ID NO. 2) |

TABLE 3

| Reaction Component | Volume (μl) |
|---|---|
| ddH₂O | 7.4 |
| PCR mixture | 10 |
| Upstream primer | 0.8 |
| (10 pmol/μl) | |
| Downstream primer | 0.8 |
| (10 pmol/μl) | |
| Template DNA | 1 |
| Total | 20 |

Herein, PCR mixture was 2×Rapid Taq Master Mix (Vazyme, Code No: P222).

TABLE 4

| | Temperature | | | Cycle |
|---|---|---|---|---|
| Step | (° C.) | Time | | (times) |
| 1 | 95 | 3 | min | — |
| 2 | 95 | 15 | sec | 34 |
| 3 | 60 | 15 | sec | |
| 4 | 72 | 1 | min | |
| 5 | 72 | 5 | min | — |

| | | | |
|---|---|---|---|
| 6 | 12 | — | Store |

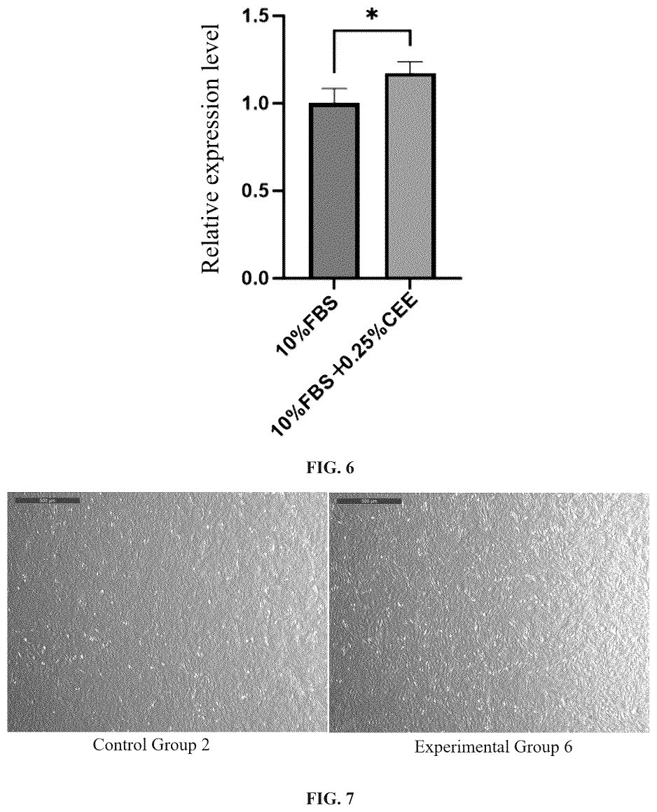

Outcomes are as shown in FIG. 6. The LF gene expression in the experimental group was significantly increased compared with that in the control group.

### Example 7 Configuration of MCF-10A Cell Medium

Formulation of MCF-10A cell proliferation medium in the present experiment is as listed in Table 5.

TABLE 5

| Group | Component | | | | | | | | |
|---|---|---|---|---|---|---|---|---|---|
| Control | DMEM | 10% | 1% | 1% | 1% non- | 20 ng/ml | 10 μg/ml | 0.5 μg/ml | Without |
| Group 2 | medium | FBS | Penicillin | Streptomycin | essential | epidermal | insulin | hydrocortisone | CEE |
| Experimental | | | (10000 | (10000 | amino acid | growth | | | 0.25% |
| Group 6 | | | U/mL) | U/mL) | | factor | | | CEE |

### Example 8 Microscopic Observation and Cell Counting of MCF-10A Cells

MCF-10A cells were seeded in six-well plates at 2.5×10⁵ cells per well. After 12 h of culture in complete medium without CEE, the control group was changed with the complete medium without CEE (Control Group 2), and the experimental group was changed with the complete medium with 0.25% CEE (Experimental Group 6). To each well of the six-well plates, 20 μl of prolactin induction solution at a concentration of 0.25 mg/ml was added. Microscopy and cell counting were performed on the third day.

Outcomes are as shown in FIG. 7. After 72 h of culture of the MCF-10A cells using the pancreatic digestion method, cell growth status was good. The experimental group and the control group were full, had a three-dimensional feeling, and exhibited a spindle shape or a triangular shape. As seen from outcomes of cell counting in FIG. 8, cells cultured with 0.25% CEE and cells in the control group without CEE did not have apparent difference in the number.

### Example 9 ELISA Detection of LF Expression in MCF-10A Cells.

MCF-10A cells were seeded in six-well plates at 2.5×10⁵ cells per well. After 12 h of culture in complete medium without CEE, the control group was changed with the complete medium without CEE (Control Group 2), and the experimental group was changed with the complete medium with 0.25% CEE (Experimental Group 6). 20 μl of prolactin induction solution at a concentration of 0.25 mg/ml was added to each well in the six-well plates. On the third day of culture, the cell medium was pipetted and centrifuged at 1,000×g for 20 min, and supernatant was taken to detect expression of bovine LF (detection steps were consistent with that for detecting the LF expression in Example 5).

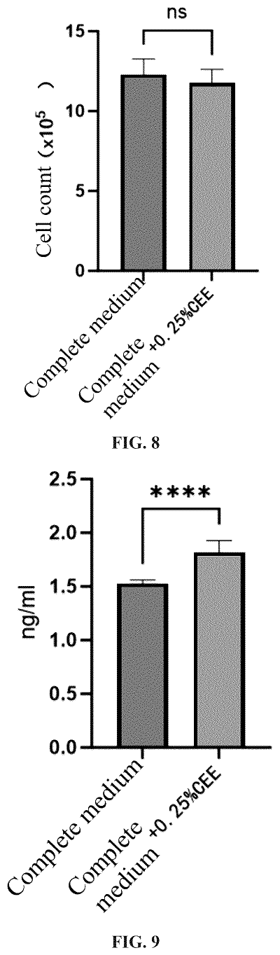

Outcomes are as shown in FIG. 9, and it can be seen that the LF content in the cells in the experimental group with 0.25% CEE was significantly higher than that in the control group. In conjunction with data in FIG. 8, when no significant proliferation occurred to cells after addition of CEE, LF yield could also be improved.

Finally, it should be explained that the various examples above are merely used for illustrating the technical solutions of the present disclosure, rather than limiting the present disclosure; while the detailed description is made to the present disclosure with reference to the preceding embodiments, those ordinarily skilled in the art should understand that they still could modify the technical solutions recited in the preceding embodiments, or make equivalent substitutions to some or all of the technical features therein; these modifications or substitutions do not make the corresponding technical solutions essentially depart from the scope of the technical solutions of the various embodiments of the present disclosure.

1. Use of a chick embryo extract in promoting synthesis of lactoferrin by mammary epithelial cells.

2. The use according to claim 1, wherein the promoting synthesis of lactoferrin by mammary epithelial cells comprises improving an expression level of lactoferrin-encoding genes and/or increasing a lactoferrin content.

3. The use according to claim 1, wherein the mammary epithelial cells are derived from mammals.

4. The use according to claim 3, wherein the mammals comprise any one selected from the group consisting of humans, cattle, sheep, camels, horses, donkeys, reindeer, alpacas, and llamas.

5. A composition for promoting synthesis of lactoferrin by mammary epithelial cells, comprising 0.25-0.5 mL of a chick embryo extract per 100 mL of a medium.

6. The composition according to claim 5, comprising 0.25 mL of the chick embryo extract per 100 mL of the medium.

7. The composition according to claim 5, wherein the medium is a mammary epithelial cell medium.

8. The composition according to claim 7, wherein the mammary epithelial cell medium comprises a DMEM medium and a fetal bovine serum.

9. A method for promoting synthesis of lactoferrin by mammary epithelial cells in vitro, using the composition according to claim 5 for culturing the mammary epithelial cells.

10. The method according to claim 9, when the chick embryo extract is added in an amount of 0.25 mL per 100 mL of the medium, a culturing duration is at least 4 days; and
when the chick embryo extract is added in an amount of 0.5 mL per 100 mL of the medium, the culturing duration is at least 6 days.
